(19) United States
(12) Patent Application Publication
(10) Pub. No.: US 2011/0046475 A1
(43) Pub. Date: Feb. 24, 2011

(54) TECHNIQUES FOR CORRECTING TEMPERATURE MEASUREMENT IN MAGNETIC RESONANCE THERMOMETRY

(76) Inventors: Benny Assif, Ramat HaSharon (IL); Charles L. Dumoulin, Cincinnati, OH (US); Richard P. Mallozzi, Middleton, MA (US); Robert D. Darrow, Scotia, NY (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/546,269

(22) Filed: Aug. 24, 2009

(51) Int. Cl. A61B 5/055

(52) U.S. Cl. 600/422

(57) ABSTRACT

Techniques for correcting temperature measurement in MR thermometry are disclosed. In particular, phase shifts that arise from factors other than temperature changes are detected, facilitating correction of temperature measurements.

### FIELD OF THE INVENTION

The present invention relates generally to magnetic resonance (MR) imaging, and more particularly, to techniques for correcting temperature measurement in MR thermometry.

### BACKGROUND OF THE INVENTION

MR imaging of internal body tissues may be used for numerous medical procedures, including diagnosis and surgery. In general terms, MR imaging starts by placing a subject in a relatively uniform, static magnetic field. The static magnetic field causes hydrogen nuclei spins to align and precess about the general direction of the magnetic field. The nuclear spins align either parallel or anti-parallel to the static magnetic field. Spins in these states have slightly different energies. Furthermore, the number of spins in the lower energy (i.e., ground) state slightly exceeds the number found in the higher energy (i.e., excited) state. This slight excess in population of the lower energy state results in a net magnetization. Radio frequency (RF) magnetic field pulses are then superimposed on the static magnetic field to cause the net magnetization to rotate into the plane perpendicular to the direction of the static magnetic field. Magnetization in this plane can give rise to an MR response signal. It is known that different tissues in the subject produce different MR response signals, and this property can be used to create contrast in an MR image. An RF receiver detects the duration, strength, and source location of the MR response signals, and such data are then processed to generate tomographic or three-dimensional images.

MR imaging can also be used effectively during a medical procedure to assist in locating and guiding medical instruments. For example, a medical procedure can be performed on a patient using medical instruments while the patient is in an MRI machine. The medical instruments may be for insertion into a patient or they may be used externally but still have a therapeutic or diagnostic effect. For instance, the medical instrument can be an ultrasonic device, which is disposed outside a patient's body and focuses ultrasonic energy to ablate or necrose tissue or other material on or within the patient's body. The MRI machine preferably produces images at a high rate so that the location of the instrument (or the focus of its effects) relative to the patient may be monitored in real-time (or substantially in real-time). The MRI machine can be used for both imaging the targeted body tissue and locating the instrument, such that the tissue image and the overlaid instrument image can help track an absolute location of the instrument as well as its location relative to the patient's body tissue.

MR imaging can further provide a non-invasive means of quantitatively monitoring in vivo temperatures. This is particularly useful in the above-mentioned MR-guided focused ultrasound (MRgFUS) treatment or other MR-guided thermal therapy where temperature of a treatment area should be continuously monitored in order to assess the progress of treatment and correct for local differences in heat conduction and energy absorption. The monitoring (e.g., measurement and/or mapping) of temperature with MR imaging is generally referred to as MR thermometry or MR thermal imaging.

Among the various methods available for MR thermometry, proton-resonance frequency (PRF) shift method is often preferred due to its excellent linearity with respect to temperature change, near-independence from tissue type, and good sensitivity. The PRF shift method is based on the phenomenon that the MR resonance frequency of protons in water molecules changes linearly with temperature. Since the frequency change is small, only −0.01 ppm/° C. for bulk water and approximately −0.0096-−0.013 ppm/° C. in tissue, the PRF shift is typically detected with a phase-sensitive imaging method in which the imaging is performed twice: first to acquire a baseline PRF phase image prior to a temperature change and then to acquire a second image after the temperature change, thereby capturing a small phase change that is proportional to the change in temperature.

A phase image, for example, may be computed from an MR image, and a temperature-difference map relative to the baseline image may be obtained by (i) subtracting, on a pixel-by-pixel basis, the phase image corresponding to the baseline from the phase image corresponding to a subsequently obtained MR image, and (ii) converting phase differences into temperature differences based on the PRF temperature dependence.

Unfortunately, changes in PRF phase images do not arise uniquely from temperature changes. Various non-temperature-related factors, such as changes in a local magnetic field due to nearby moving metal, magnetic susceptibility changes in a patient's body due to breathing or movement, and magnet or shim drifts can all lead to confounding changes in phase measurement that may render a phase-sensitive temperature measurement invalid. The changes in magnetic field associated with magnet drift and patient motion are often severe enough to render temperature measurements made using the above-mentioned phase-sensitive approach useless. This effect becomes quite significant when temperature change is monitored over a long time, such as during a long treatment procedure. As the elapsed time between the initial baseline phase image and the actual temperature measurement increases, concurrent (and non-temperature-related) changes in magnetic field are more likely to occur, causing erroneous temperature measurement.

In view of the foregoing, it may be understood that there are significant problems and shortcomings associated with current PRF method of MR thermometry.

### SUMMARY

Embodiments of the present invention measure and compensate for phase shifts that arise from factors other than temperature changes, facilitating correction of temperature measurements in MR thermometry. In particular, one or more micro-coils may be placed near the region being monitored. These coils may be filled with a substance (e.g., oil) whose MR signal is temperature invariant, or may be filled with, e.g., water and placed in a region whose temperature is constant or known. The MR frequency detected by the micro-coils is unaffected by temperature, and as a result, the frequency measured at each micro-coil may be used to compute the phase background for the thermal imaging acquisition.

In one particular exemplary embodiment, a method of correcting PRF-based MR temperature measurement may comprise the step of detecting at least one first MR response of one or more micro-coils located in or near a region of interest. The detection is performed approximately when a first PRF image of the region of interest is acquired. The method may also comprise the step of detecting at least one second MR response of the micro-coil(s) approximately when a second PRF image of the region of interest is acquired and, for example, determining a temperature-invariant difference between the second MR response(s) and the first MR response(s), where the temperature-invariant difference is caused by factors unrelated to a temperature change in or near the region of interest. In some embodiments, the method may additionally include correcting, based on the temperature-invariant difference, a temperature measurement of the region of interest made from the second PRF image and the first PRF image.

In another particular exemplary embodiment, a system for correcting PRF-based MR temperature measurement may comprise an MRI unit and one or more micro-coils configured to generate MR response signals in response to the MRI unit; the micro-coil(s) are sufficiently small to be placed in or near a region of interest. The system may further comprise a control module in communication with the MRI unit and the one or more micro-coils. The control module may be configured to cause at least one first MR response of the one or more micro-coils to be detected approximately when the MRI unit acquires a first PRF image of the region of interest, and cause at least one second MR response of the one or more micro-coils to be detected approximately when the MRI unit acquires a second PRF image of the region of interest. The system may additionally comprise a processing module for determining a temperature-invariant difference between the second MR response(s) and the first MR response(s), where the temperature-invariant difference is caused by factors unrelated to a temperature change in or near the region of interest. The processing module may correct, based on the temperature-invariant difference, a temperature measurement of the region of interest made from the second PRF image and the first PRF image.

In yet another particular exemplary embodiment, a computer-readable medium storing computer-executable codes for causing at least one processor to correct PRF-based MR temperature measurement may comprise computer-executable code adapted to detect at least one first MR response of one or more micro-coils located in or near a region of interest, where the detection is performed approximately when a first PRF image of the region of interest is acquired. The computer-readable medium may also comprise computer-executable code adapted to detect at least one second MR response of the one or more micro-coils approximately when a second PRF image of the region of interest is acquired. The computer-readable medium may further comprise computer-executable code adapted to determine a temperature-invariant difference between the second MR response(s) and the first MR response(s), where the temperature-invariant difference are caused by factors unrelated to a temperature change in or near the region of interest. The computer-readable medium may additionally comprise computer-executable code adapted to correct, based on the temperature-invariant difference, a temperature measurement of the region of interest made from the second PRF image and the first PRF image.

The present invention will now be described in more detail with reference to exemplary embodiments thereof as shown in the accompanying drawings. While the present invention is described below with reference to exemplary embodiments, it should be understood that the present invention is not limited thereto. Those of ordinary skill in the art having access to the teachings herein will recognize additional implementations, modifications, and embodiments, as well as other fields of use, which are within the scope of the present invention as described herein, and with respect to which the present invention may be of significant utility.

### BRIEF DESCRIPTION OF THE DRAWINGS

In order to facilitate a fuller understanding of the present invention, reference is now made to the accompanying drawings, in which like elements are referenced with like numerals. These drawings should not be construed as limiting the present invention, but are intended to be exemplary only.

FIG. 1 shows an exemplary MRI system in or for which the techniques for correcting temperature measurement in accordance with the present invention may be implemented.

FIG. 2 shows an imaging region in which exemplary micro-coils are deployed in accordance with an embodiment of the present invention.

FIG. 3 shows an exemplary micro-coil for correcting temperature measurement in MR thermometry in accordance with an embodiment of the present invention.

FIG. 4 shows a flow chart illustrating an exemplary method for correcting temperature measurement in MR thermometry in accordance with an embodiment of the present invention.

FIG. 5 shows an exemplary configuration of micro-coils for correcting temperature measurement in accordance with an embodiment of the present invention.

FIG. 6 shows a block diagram illustrating an exemplary system for correcting temperature measurement in MR thermometry in accordance with an embodiment of the present invention.

### DETAILED DESCRIPTION

Embodiments of the present invention improve the utility and robustness of MR thermometry, as described below, to measure and compensate for local magnetic field changes or PRF phase shifts that arise from factors other than temperature changes.

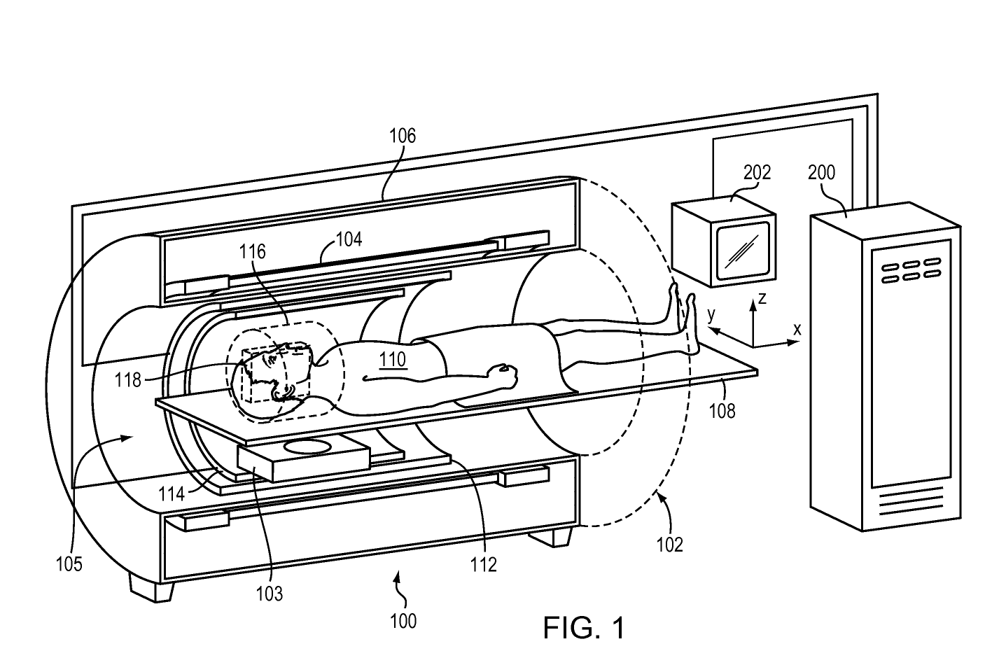

FIG. 1 shows an exemplary MRI system 100 in or for which the techniques for correcting temperature measurement in accordance with the present invention may be implemented. The illustrated MRI system 100 comprises an MRI machine 102 with a magnet bore 105. If an MR-guided procedure is being performed, a medical device 103 may be disposed within the bore of the MRI machine 102. Since the components and operation of the MRI machine are well-known in the art, only some basic components helpful in the understanding of the system 100 and its operation will be described herein.

The MRI machine 102 typically comprises a cylindrical electromagnet 104, which generates a static magnetic field within a bore 105 of the electromagnet 104. The electromagnet 104 generates a substantially homogeneous magnetic field within an imaging region 116 inside the magnet bore 105. The electromagnet 104 may be enclosed in a magnet housing 106. A support table 108, upon which a patient 110 lies, is disposed within the magnet bore 105. A region of interest 118 within the patient 110 may be identified and positioned within the imaging region 116 of the MRI machine 102.

A set of cylindrical magnetic field gradient coils 112 may also be provided within the magnet bore 105. The gradient coils 112 also surround the patient 110. The gradient coils 112 can generate magnetic field gradients of predetermined magnitudes, at predetermined times, and in three mutually orthogonal directions within the magnet bore 105. With the field gradients, different spatial locations can be associated with different precession frequencies, thereby giving an MR image its spatial resolution. An RF transmitter coil 114 surrounds the imaging region 116 and the region of interest 118. The RF transmitter coil 114 emits RF energy in the form of a magnetic field into the imaging region 116, including into the region of interest 118.

The RF transmitter coil 114 can also receive MR response signals emitted from the region of interest 118. The MR response signals are amplified, conditioned and digitized into raw data using an image processing system 200, as is known by those of ordinary skill in the art. The image processing system 200 further processes the raw data using known computational methods, including fast Fourier transform (FFT), into an array of image data. The image data may then be displayed on a monitor 202, such as a computer CRT, LCD display or other suitable display.

The medical device 103 may also be placed within the imaging region 116 of the MRI machine 102. In the example shown in FIG. 1, the medical device 103 may be an ultrasonic ablation instrument used for ablating tissue such as fibroids or cancerous (or non-cancerous) tissue, for breaking up occlusion within vessels, or for performing other treatment of tissues on or within the patient 110. In fact, the medical device 103 can be any type of medical instrument including, without limitation, a needle, catheter, guidewire, radiation transmitter, endoscope, laparoscope, or other instrument. In addition, the medical device 103 can be configured either for placement outside the patient 110 or for insertion into the patient body.

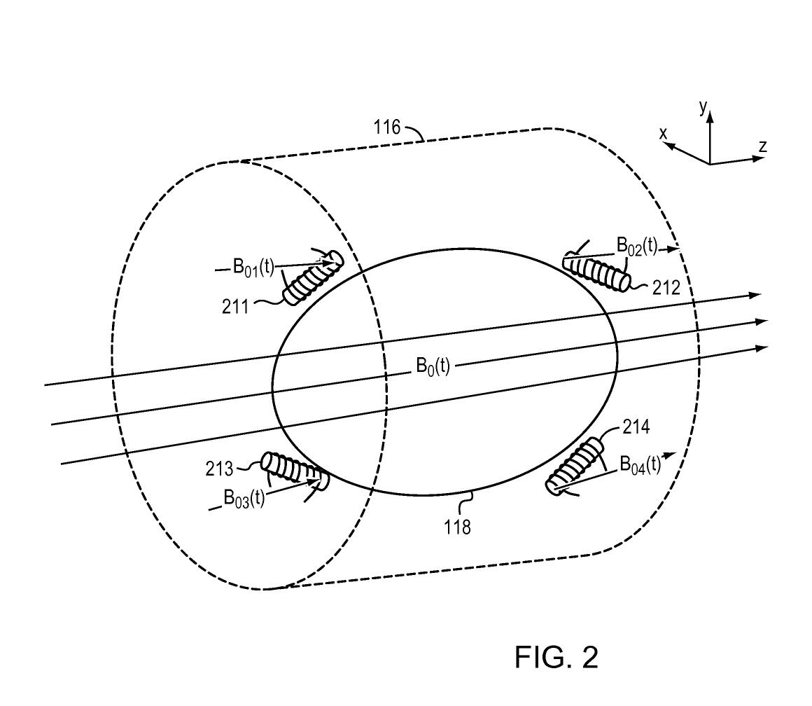

The imaging region 116 (including the region of interest 118) is enlarged in FIG. 2, to illustrate an exemplary deployment of micro-coils in accordance with an embodiment of the present invention.

During MR thermal imaging (or any medical procedure involving MR temperature mapping) of the region of interest 118, a background magnetic field B₀(t) may change due to various factors unrelated to changes in temperature. As a result, an initial baseline PRF phase image acquired prior to the change in the background magnetic field becomes unreliable. To solve this problem, one or more MR pick-up coils, such as micro-coils 211, 212, 213, and 214, may be deployed in or near the region of interest 118 to detect changes in local magnetic fields B₀₁(t), B₀₂(t), B₀₃(t), and B₀₄(t), respectively. The MR pick-up coils are preferably fixed in space and located within the imaging region 116. For example, the micro-coils may be attached to a rigid frame that is independent of any other structure in the MR system. Alternatively, the micro-coils may be incorporated into one of the imaging coils or in the medical device 103. Attachment of the micro-coils to the patient is also possible as long as patient motion does not affect the magnetic field in the vicinity of the micro-coils and the micro-coils are electrically isolated from the patient. In response to MR pulse sequences, the micro-coils 211, 212, 213, and 214 may generate MR response signals which provide information on local magnetic fields B₀₁(t), B₀₂(t), B₀₃(t), and B₀₄(t) at the respective locations of the micro-coils. Depending on the type of micro-coils used, the detected changes in local magnetic fields either do not contain temperature-dependent components or such components may be filtered out. The local magnetic field data may then be used to correct MR temperature measurement, for example, by correcting the baseline PRF phase image. According to one embodiment of the present invention, when a single micro-coil such as the coil 211 is deployed, the detected local magnetic field B₀₁(t) or related data can provide a zero-th order (i.e., spatially uniform) correction to the MR temperature measurement. According to another embodiment of the present invention, four or more micro-coils may be deployed in a non-coplanar fashion, and the resulting local magnetic field data may provide sufficient information for both zero-th and first-order corrections to the MR temperature measurement in three dimensions. For example, five or more micro-coils may be able to provide a zero-th order, a first order, and at least a partial second order corrections to the MR temperature measurement.

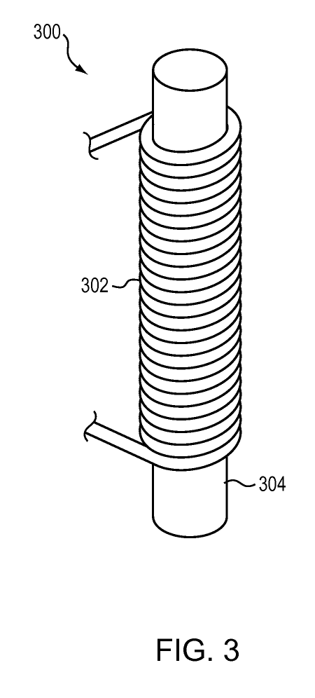

FIG. 3 shows an exemplary micro-coil 300 for correcting temperature measurement in MR thermometry in accordance with an embodiment of the present invention. The micro-coil 300 may comprise a hollow-core solenoid 302 and a tube 304 inserted into the windings of the solenoid 302. The solenoid 302 may be made of a conductive material (e.g., metal wire). The tube 304 may be filled with a MR-sensitive substance, preferably in liquid form, such as oil or water. According to one embodiment of the present invention, the tube 304 is filled with oil or other non-aqueous fluid whose MR response signal is insensitive to temperature change. If the tube 304 is filled with water or a water-based substance, MR response signals detected with the micro-coil 300 will include temperature-dependent components which should be accounted for in the correction of MR temperature measurement.

According to embodiments of the present invention, the micro-coil 300 may have the same design or one similar to the MR tracking coils used in MRgFUS or related fields. Preferably, the micro-coil 300 is sufficiently small that its MR sensitivity drops quickly with distance from its location or, in other words, it only detects the MR response signal from excited nuclei very close to itself. Thus, the micro-coil 300 can represent a certain point location inside the MR bore. On the other hand, the micro-coil 300 should also be large enough to produce sufficiently strong signals. According to one particular embodiment of the present invention, the solenoid 302 is approximately 5 mm in length and has a 1.5 mm inner diameter, and the tube 304 is approximately 7 mm in length and has a 1.5 mm outer diameter so that it fits within the solenoid 302.

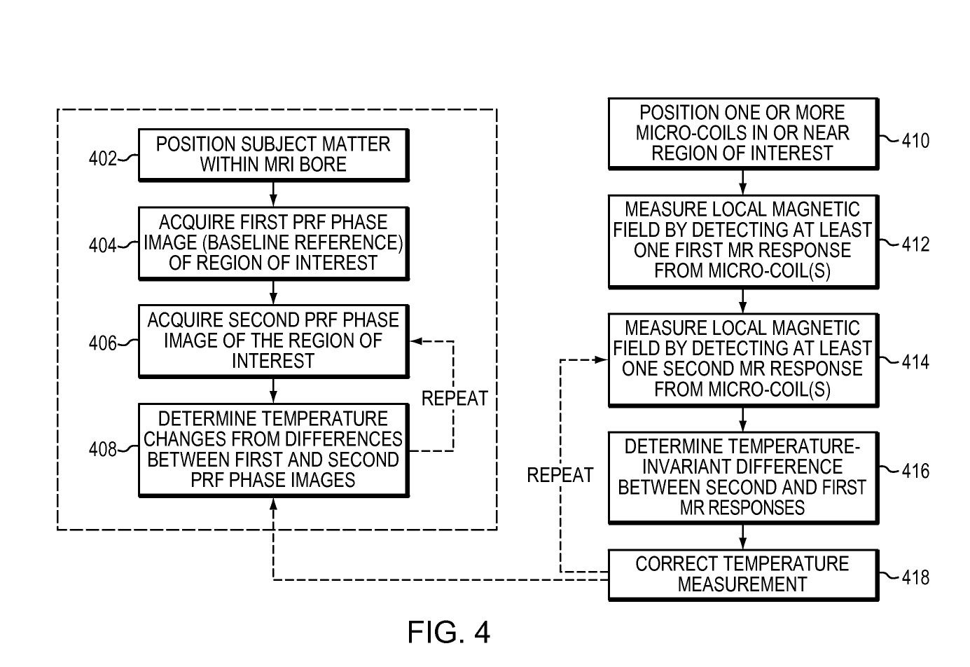

FIG. 4 shows a flow chart illustrating an exemplary method for correcting temperature measurement in MR thermometry in accordance with an embodiment of the present invention. In step 402, a subject matter such as a human body, may be positioned within a bore of an MRI machine. A region of interest (ROI) in the subject matter may be identified for purposes of MR temperature measurement, that is, MR thermal imaging or temperature mapping. For example, the region of interest may be a portion of a human body, such as the head region (118) as shown in FIG. 1. In an MR-guided medical procedure, the region of interest may be or include a particular portion of a human body upon which the procedure is performed. For instance, in an MRgFUS procedure, the region of interest may include a general tissue area into which ultrasonic energy is to be focused.

At approximately the same time as step 402, one or more magnetic-field-sensing devices may be positioned in or near the region of interest (step 410). The magnetic-field-sensing device(s) may preferably include one or more MR pick-up coils such as micro-coils, although other types of magnetic field sensors (e.g., Hall effect sensors or Hall probes) may instead be used. The micro-coils are typically deployed close to ROI locations where accurate temperature measurement is desirable. Where multiple micro-coils are deployed, they can be separated from one another by a small distance, for example, a few centimeters.

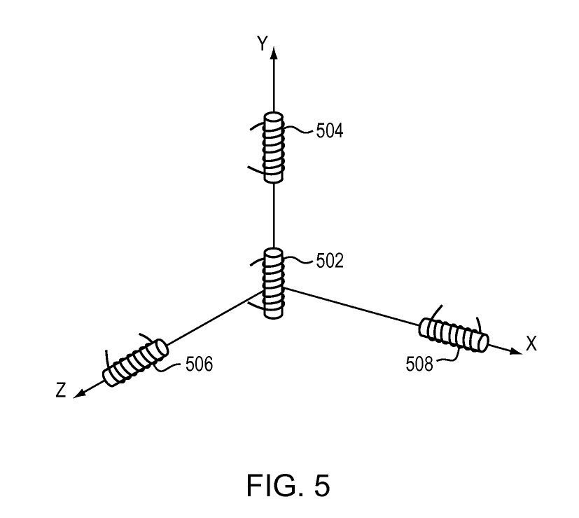

Assuming the micro-coils are sufficiently small and can be treated as geometric points within the MR field of view, the number of micro-coils and their relative positions may affect the correction they can provide for MR temperature measurement. If only a single micro-coil is deployed in or near the region of interest, the local magnetic field change detected at that micro-coil is uniformly applied across the entire region of interest. If four or more micro-coils are deployed, a three-dimensional local magnetic field correction may be obtained, provided that these four or more micro-coils are not placed in the same plane. FIG. 5 shows an exemplary configuration of four micro-coils (502, 504, 506, and 508) for correcting temperature measurements in accordance with an embodiment of the present invention. In this example, the micro-coil 502 is placed at the origin of a Cartesian coordinate system, while the micro-coils 504, 506, and 508 are placed on the X, Y, and Z axis, respectively. Preferably these micro-coils are positioned in a non-coplanar fashion, i.e., if any three micro-coils are selected, the fourth will be outside of the plane defined by the selected three. Of course, numerous other variations exist for configuring the micro-coils to meet the condition of non-coplanarity.

Referring back to FIG. 4, an MR thermal imaging process may start in step 404 when a first PRF phase image of the region of interest is acquired. This first PRF phase image essentially captures a distribution of proton-resonance frequencies in the region of interest, and may serve as a baseline reference for subsequent PRF-based MR temperature measurements.

In step 412, approximately when the first PRF phase image of the region of interest is acquired or around substantially the same time as step 404, local magnetic field(s) may be measured by detecting at least one first MR response from the one or more micro-coils. That is, MR response signals from the micro-coil(s) in response to MR pulse sequences may be processed to determine a local magnetic field or PRF phase field at each micro-coil location. According to embodiments of the present invention, a number of MR pulse sequences or combinations thereof may be used for the micro-coils to measure local magnetic fields or PRF phase fields, as will be explained below.

Next, in step 406, a second PRF phase image of the region of interest may be acquired. This acquisition step, together with the prior acquisition step 404, may be part of an MR thermal imaging process. The second PRF phase image essentially captures the distribution of proton-resonance frequencies in the region of interest at the time of the acquisition step 406. Depending on whether temperature has changed in the region of interest since the baseline reference was acquired in step 404, the second PRF phase image may or may not be substantially different from the first PRF phase image.

Then, in step 408, temperature changes in the region of interest between steps 404 and 406 may be determined from PRF phase or frequency differences reflected in the second PRF phase image as compared to the first PRF phase image. A temperature map, reflecting absolute temperature values or relative temperature changes in the region of interest, may be generated based at least in part on the differences between the second and the first PRF phase images. As part of a continuous MR thermal imaging process, steps 406 and 408 may be repeated to continuously monitor temperature changes in the region of interest, for example, by updating the temperature map.

In step 414, approximately when the second PRF phase image of the region of interest is acquired or around substantially the same time as step 406, local magnetic field(s) may be measured again by detecting at least one second MR response from the micro-coil(s). This step captures changes in the local magnetic fields or PRF phase fields at the micro-coil locations which occurred between approximately the time of acquisition of the first PRF phase image and the time of acquisition of the second PRF phase image.

As mentioned earlier, a variety of MR pulse sequences may be useful for measuring local magnetic fields with the micro-coils.

According to one embodiment of the present invention, multiplexed pulse sequences, such as those for exciting MR tracking coils, may use four RF excitations to acquire four data sets from each micro-coil in the presence of a frequency-encoding magnetic field gradient. The four data sets can then be solved to determine X, Y, and Z coordinates of the micro-coil as well as the local magnetic field at the micro-coil location.

According to another embodiment of the present invention, a spectral acquisition method may be employed, wherein one RF pulse may be used to cause one MR response to be detected from each of the one or more micro-coils in the absence of an applied magnetic field gradient. Then, the local magnetic field may be calculated based on the detected MR responses without determining the coordinates of the micro-coils. However, the locations of micro-coils may be known or determined by other means. For example, the above-described method of MR tracking with gradients can be applied to the first acquisition to determine the micro-coil locations, and it may be assumed that the micro-coils do not move after the first acquisition. Alternatively, the location of the micro-coils could be determined based on the location of the rigid frame or structure to which the micro-coils are attached.

According to yet another embodiment of the present invention, a phase-sensitive acquisition may be performed to capture local PRF phase changes correlated to local magnetic field changes at the micro-coil locations. That is, local PRF phase images at the micro-coil locations may be acquired each time a PRF phase image of the region of interest is acquired. Since the temperatures at the micro-coil locations are constant or known, the local PRF phase images can provide data on non-temperature-related changes in the background field.

According to still another embodiment of the present invention, a pulse sequence for MR imaging may be used to determine the coordinates of a micro-coil if the scan plane is selected to include the micro-coil.

According to further embodiments of the present invention, both location and magnetic field measurements may be obtained through a hybrid pulse sequence in which some or all of the above-described multiplexed MR tracking, spectral acquisition, phase sensitive acquisition, and/or MR imaging methods are applied sequentially, either during the same MR thermal imaging step or in consecutive steps.

Referring again to FIG. 4, in step 416, a temperature-invariant difference between the second MR response(s) and the first MR response(s) may be determined. It should be noted that some of the differences between first and second MR responses may be caused by temperature-related factors. Since a goal of the present invention is to reduce or remove only non-temperature-related interferences with the PRF method of MR thermometry, temperature-induced phase or magnetic field changes picked up by the micro-coils should be filtered or discounted. If the micro-coils are filled with oil or a non-aqueous substance, the MR responses from the micro-coils may be so insensitive to temperature change that the phase or magnetic field changes picked up by the micro-coils may already be considered temperature-invariant. If, however, the micro-coils are filled with water or a water-based substance, their MR responses will change with temperature. The temperature-dependent components of those MR responses may be determined or estimated based on independently measured temperature or temperature change at each micro-coil location.

In step 418, the temperature measurement data obtained in step 408 may be corrected. According to one embodiment of the present invention, zero-th and/or first-order PRF phase changes may be calculated from the measurement of local magnetic field(s) and then applied to the baseline PRF phase image of the region of interest. In particular, depending on the number of micro-coils used in the field measurement, zero-th and/or first order phase change coefficients may be calculated and used as a basis of determining phase correction for each pixel within the baseline PRF phase image.

According to an alternative embodiment of the present invention, the (effective) values of electrical currents that are applied to B0, X, Y, Z and/or higher order magnet shims of the MR system may be calculated from the local magnetic field measure data. Here, B0 refers to the magnet shim generating the main, static magnetic field through the MR bore, and X, Y, and Z refer to magnet shims generating the magnetic field gradients in the three orientations. Since the effective currents applied to these magnet shims correct for magnetic field drifts, it can be assumed that the MR thermal imaging measurement based on the corrected magnetic field does not suffer from background drifts, thereby obviating the need for a mathematical correction on the baseline PRF phase image itself. It is well known to those skilled in the art that changing the center frequency of the MR transceiver system is equivalent to changing the main magnetic field strength, B0. It should be noted that this closed-loop correction of shim currents could have applications beyond MR thermal imaging and may be useful for MR imaging and spectroscopy methods requiring improved field stability.

By now, it should be appreciated that steps 402-408 as illustrated in FIG. 4 (enclosed in a dashed line box) represent a conventional PRF shift method of MR thermometry. Steps 410-418, when performed in proper timing relation with respect to steps 402-408, can improve the conventional PRF shift method of MR thermometry by providing correction or calibration to the temperature measurement.

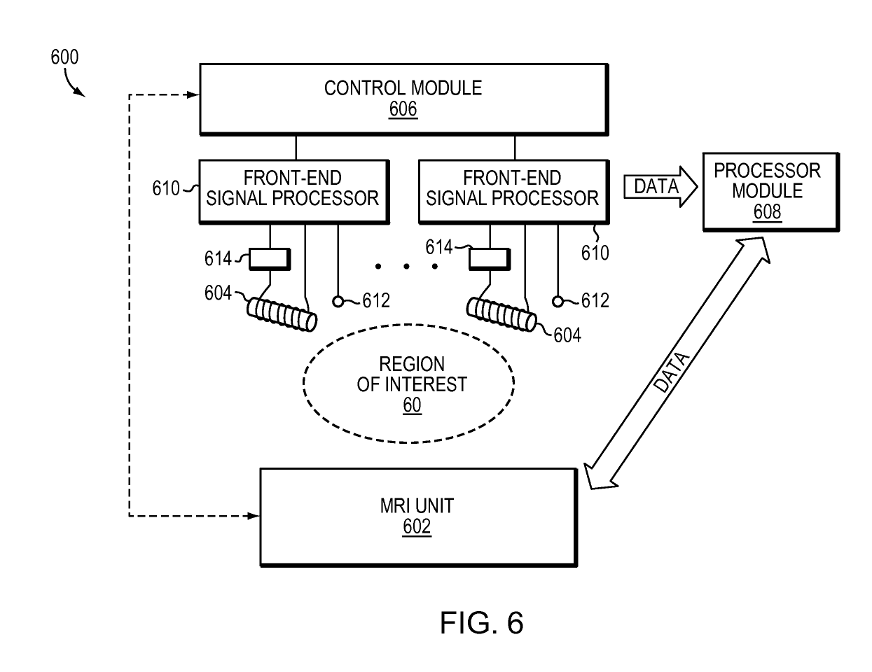

FIG. 6 shows a block diagram illustrating an exemplary system 600 for correcting temperature measurement in MR thermometry in accordance with an embodiment of the present invention. The system 600 may comprise an MRI unit 602 whose imaging field covers a region of interest 60. The MRI unit 602 may be configured for thermal imaging of the region of interest 60 based on the PRF shift method. One or more micro-coils 604 may be placed in or near the region of interest 60 to measure local magnetic field changes occurring at each micro-coil location by generating MR response signals in response to pulse sequences from the MR unit 602. Optionally, the micro-coils 604 may include or be equipped with conventional tuning and matching capacitors 614 to optimize their signal-to-noise ratio (SNR). These capacitors 614 improve the SNR by forming LC resonant circuits with the micro-coils 604 and facilitating adjustment of LC constants or impedances of the LC resonant circuits for improved signal sensitivities. The micro-coils 604 may also include or be equipped with conventional decoupling circuitry to disable the micro-coils 604 during RF transmit (provided that a larger transmit coil is used), or the micro-coils 604 may be equipped with conventional transmit/receive hardware that permits the micro-coils 604 to have both transmit and receive functions. The decoupling circuitry and the transmit/receive hardware may be implemented as part of a front-end signal processing device 610 or a control module 606. According to some embodiments of the present invention, one or more temperature sensors 612 may also be provided to independently monitor temperatures at the micro-coil location(s). The control module 606 in communication with the MRI unit 602 may coordinate the micro-coils' measurement of local magnetic fields (and/or temperatures) with image acquisitions by the MRI unit 602. The front-end signal-processing device 610 and/or similar device(s) may receive the MR response signals from the micro-coil(s) 604 and convert the signals to data. The front-end signal-processing device 610 may also receive temperature-measurement signals or data from the temperature sensors 612. These data, together with image acquisition data from the MRI unit 602, may be processed by a processor module 608 where the data related to local magnetic field changes (and/or local temperature changes) are used to correct thermal imaging of the region of interest 60.

It should be noted that, although portions of the system 600 have been illustrated as discrete components in FIG. 6, some of these components (e.g., control module 606, processor module 608, and front-end signal-processing device 610) may be combined with one another and/or implemented as integral part(s) of the MRI unit 602. Other variations exist for configuring the system 600 as can be appreciated by those skilled in the art.

While the foregoing description includes many details and specificities, it is to be understood that these have been included for purposes of explanation only, and are not to be interpreted as limitations of the present invention. It will be apparent to those skilled in the art that other modifications to the embodiments described above can be made without departing from the spirit and scope of the invention. Accordingly, such modifications are considered within the scope of the invention as intended to be encompassed by the following claims and their legal equivalents.

What is claimed is:

1. A method of correcting proton resonance frequency (PRF) based magnetic resonance (MR) temperature measurement, the method comprising the steps of:
detecting at least one first MR response of one or more micro-coils located in or near a region of interest, the detection being performed approximately when a first PRF image of the region of interest is acquired;
detecting at least one second MR response of the one or more micro-coils approximately when a second PRF image of the region of interest is acquired;
determining a temperature-invariant difference between the at least one second MR response and the at least one first MR response, the temperature-invariant difference being caused by factors unrelated to a temperature change in or near the region of interest; and
correcting, based on the temperature-invariant difference, a temperature measurement of the region of interest made from the second PRF image and the first PRF image.

2. The method of claim 1, wherein (i) the one or more micro-coils consist of a single micro-coil, and (ii) the temperature-invariant difference determined from the at least one second MR response and the at least one first MR response of the single micro-coil provides a spatially uniform correction to the first PRF image.

3. The method of claim 1, wherein (i) the one or more micro-coils consist of four micro-coils arranged in a non-coplanar fashion, and (ii) the temperature-invariant difference determined from the at least one second MR response and the at least one first MR response of the four micro-coils provides a zero-th order and a first order corrections to the first PRF image.

4. The method of claim 1, wherein (i) the one or more micro-coils consist of five or more micro-coils arranged in a non-coplanar fashion, and (ii) the temperature-invariant difference determined from the at least one second MR response and the at least one first MR response of the five or more micro-coils provides a zero-th order, a first order, and at least a partial second order corrections to the first PRF image.

5. The method of claim 1, wherein the one or more micro-coils are filled with a substance that provides temperature-insensitive MR responses.

6. The method of claim 5, wherein the one or more micro-coils are filled with oil or other non-aqueous fluid.

7. The method of claim 1, wherein the one or more micro-coils are filled with water or water-based substance, and wherein temperature(s) at the locations of the micro-coil(s) are constant.

8. The method of claim 1, wherein the one or more micro-coils are filled with water or water-based substance and temperature(s) at the locations of the micro-coil(s) are known, and further comprising adjusting for a temperature-dependent component of the second MR response in determining the temperature-invariant difference between the at least one second MR response and the at least one first MR response.

9. The method of claim 1, further comprising:
independently determining temperature(s) at the locations of the micro-coil(s) with a non-MR-based method; and
calculating the temperature-invariant difference between the second MR response and the first MR response based at least in part on the determined temperature(s).

10. The method of claim 1, further comprising:
determining a local PRF image change in or near the region of interest based on the detection of the at least one first MR response and the detection of the at least one second MR response, the local PRF image change being correlated to a local magnetic field.

11. The method of claim 1, further comprising:
determining a local magnetic field in or near the region of interest, based on the detection of the at least one first MR response and/or the detection of the at least one second MR response, according to one or more of:
(a) an MR tracking method of detecting four MR responses from each of one or more micro-coils and then calculating coordinates of each of one or more micro-coils and the local magnetic field based on the detected MR responses; or
(b) a spectral acquisition method of detecting one MR response from each of the one or more micro-coils in the absence of an applied magnetic field gradient and then calculating the local magnetic field based on the detected MR responses without calculating the coordinates of the one or more micro-coils; or
(c) a phase-sensitive acquisition method of detecting a change in PRF phase images correlated with the local magnetic field change; or
(d) an MR imaging method of acquiring one or more MR images wherein each scan plane includes the one or more micro-coils; or
(e) a hybrid method combining at least two of (a), (b), (c) or (d).

12. The method of claim 1, further comprising:
correcting the first PRF image based on the determined temperature-invariant difference, such that the corrected first PRF image provides an updated baseline reference for the second PRF phase image.

13. The method of claim 1, further comprising:
determining MR shim currents from at least one of the at least one first MR response and the at least one second MR response; and
determining a change in magnetic field based on the MR shim currents.

14. The method of claim 1, further comprising:
correcting the second PRF image based on the determined temperature-invariant difference, such that the temperature measurement is less affected by magnetic field changes unrelated to the temperature change.

15. The method of claim 1, wherein the temperature measurement comprises an MR thermal image or temperature map.

16. A system for correcting proton resonance frequency (PRF) based magnetic resonance (MR) temperature measurement, the system comprising:
an MRI unit;
one or more micro-coils configured to generate MR response signals in response to the MRI unit, the one or more micro-coils being sufficiently small to be placed in or near a region of interest;
a control module in communication with the MRI unit and the one or more micro-coils, and configured to:
cause at least one first MR response of the one or more micro-coils to be detected approximately when the MRI unit acquires a first PRF image of the region of interest, and
cause at least one second MR response of the one or more micro-coils to be detected approximately when the MRI unit acquires a second PRF image of the region of interest; and
a processing module configured to:
determine a temperature-invariant difference between the at least one second MR response and the at least one first MR response, the temperature-invariant difference being caused by factors unrelated to a temperature change in or near the region of interest, and
correct, based on the temperature-invariant difference, a temperature measurement of the region of interest made from the second PRF image and the first PRF image.

17. The system of claim 16, wherein (i) the one or more micro-coils consist of a single micro-coil, and (ii) the temperature-invariant difference determined from the at least one second MR response and the at least one first MR response of the single micro-coil provides a spatially uniform correction to the first PRF image.

18. The system of claim 16, wherein (i) the one or more micro-coils consist of four micro-coils arranged in a non-coplanar fashion, and (ii) the temperature-invariant difference determined from the at least one second MR response and the at least one first MR response of the four micro-coils provides a zero-th order and a first order corrections to the first PRF image.

19. The system of claim 16, wherein (i) the one or more micro-coils consist of five or more micro-coils arranged in a non-coplanar fashion, and (ii) the temperature-invariant difference determined from the at least one second MR response and the at least one first MR response of the five or more micro-coils provides a zero-th order, a first order, and at least a partial second order corrections to the first PRF image.

20. The system of claim 16, wherein the one or more micro-coils are filled with a substance that provides temperature-insensitive MR responses.

21. The system of claim 20, wherein the one or more micro-coils are filled with oil or other non-aqueous fluid.

22. The system of claim 16, wherein the one or more micro-coils are filled with water or water-based substance, and wherein temperature(s) at the locations of the micro-coil(s) are constant.

23. The system of claim 16, wherein: (i) the one or more micro-coils are filled with water or water-based substance and temperature(s) at the locations of the micro-coil(s) are known, and (ii) the processing module is further configured to adjust for a temperature-dependent component of the second MR response in determining the temperature-invariant difference between the at least one second MR response and the at least one first MR response.

24. The system of claim 16, further comprising:
one or more front-end signal processors to receive the MR response signals from the one or more micro-coils.

25. The system of claim 16, further comprising:
tuning and matching capacitors coupled to the one to or more micro-coils to improve signal-to-noise ratio of the MR response signals.

26. The system of claim 16, further comprising:
decoupling circuitry to disable the one or more micro-coils during an operation of the MRI unit.

27. The system of claim 16, further comprising:
at least one temperature sensor to independently monitor temperatures at the locations of the one or more micro-coils.

28. A computer-readable medium storing computer-executable codes for causing at least one processor to correct proton resonance frequency (PRF) based magnetic resonance (MR) temperature measurement, the computer readable medium comprising:
computer-executable code adapted to detect at least one first MR response of one or more micro-coils located in or near a region of interest, the detection being performed approximately when a first PRF image of the region of interest is acquired;
computer-executable code adapted to detect at least one second MR response of the one or more micro-coils approximately when a second PRF image of the region of interest is acquired;
computer-executable code adapted to determine a temperature-invariant difference between the at least one second MR response and the at least one first MR response, the temperature-invariant difference being caused by factors unrelated to a temperature change in or near the region of interest; and
computer-executable code adapted to correct, based on the temperature-invariant difference, a temperature measurement of the region of interest made from the second PRF image and the first PRF image.

29. The computer-readable medium of claim 28, further comprising:
computer-executable code adapted to independently determine temperature(s) at the locations of the micro-coil(s) with a non-MR-based method; and
computer-executable code adapted to calculate the temperature-invariant difference between the second MR response and the first MR response based at least in part on the determined temperature(s).

30. The computer-readable medium of claim 28, further comprising:
computer-executable code adapted to determine a local PRF image change in or near the region of interest based on the detection of the at least one first MR response and the detection of the at least one second MR response, the local PRF image change being correlated to a local magnetic field.

31. The computer-readable medium of claim 28, further comprising:
computer-executable code adapted to determine a local magnetic field in or near the region of interest, based on the detection of the at least one first MR response and/or the detection of the at least one second MR response, according to one or more of:
(a) an MR tracking method of detecting four MR responses from each of one or more micro-coils and then calculating coordinates of each of one or more micro-coils and the local magnetic field based on the detected MR responses; or
(b) a spectral acquisition method of detecting one MR response from each of the one or more micro-coils in the absence of an applied magnetic field gradient and then calculating the local magnetic field based on the detected MR responses without calculating the coordinates of the one or more micro-coils; or
(c) a phase-sensitive acquisition method of detecting a change in PRF phase images correlated with the local magnetic field change; or
(d) an MR imaging method of acquiring one or more MR images wherein each scan plane includes the one or more micro-coils; or
(e) a hybrid method combining at least two of (a), (b), (c) or (d).

32. The computer-readable medium of claim 28, further comprising:
computer-executable code adapted to correct the first PRF image based on the determined temperature-invariant difference, such that the corrected first PRF image provides an updated baseline reference for the second PRF phase image.

33. The computer-readable medium of claim 28, further comprising:
computer-executable code adapted to determine MR shim currents from at least one of the at least one first MR response and the at least one second MR response; and
computer-executable code adapted to determine a change in magnetic field based on the MR shim currents.

34. The computer-readable medium of claim 28, further comprising:
computer-executable code adapted to correct the second PRF image based on the determined temperature-invariant difference, such that the temperature measurement is less affected by magnetic field changes unrelated to the temperature change.
